(19) United States
(12) Patent Application Publication
(10) Pub. No.: US 2006/0024710 A1
(43) Pub. Date: Feb. 2, 2006

(54) Method and device for identifying germs

(76) Inventors: Tilo Weiss, Duesseldorf (DE); Stefan Stumpe, Duesseldorf (DE); Friedhelm Siepmann, Essen (DE); Andreas Thuenchen, Wuppertal (DE); Frank Wienhausen, Ochtrup (DE); Andreas Katerkamp, Melsungen (DE); Michael Heinzel, Bonn (DE); Roland Breves, Mettmann (DE); Mirko Weide, Duesseldorf (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/155,457

(22) Filed: Jun. 16, 2005

Related U.S. Application Data
(63) Continuation of application No. PCT/EP03/13567, filed on Dec. 2, 2003.

(30) Foreign Application Priority Data
Dec. 17, 2002 (DE) 102 59 302.7

(51) Int. Cl. C12Q 1/68; G01N 33/554; G01N 33/569; C12Q 1/04

(52) U.S. Cl. 435/6; 435/7.32; 435/34

(57) ABSTRACT

A method for quantitative and/or qualitative determination of germs in a liquid sample comprises the steps of: (1) passing the sample through a filter thereby depositing the germs on a major portion of the filter so that a minor portion of the filter is free of germ deposits; (2) applying a fluorescent label to at least a portion of the deposited germs; (3) determining the presence and/or the amount of labeled germs by fluorescent reflection photometry. The method according to the present invention is suitable, for example, for quantitative and/or qualitative determination of germs in foodstuffs, surfactant-containing products such as washing and cleaning agents, surface treatment agents, dispersion products, cosmetics, hygiene products and personal care products, pharmaceuticals, adhesives, coolant lubricants, coatings and coating coagulations, as well as raw materials and starting materials for the aforesaid products.

### CROSS REFERENCE TO RELATED APPLICATIONS

This application is a continuation under 35 U.S.C. § 365(c) and 35 U.S.C. § 120 of international application PCT/EP2003/013567, filed Dec. 2, 2003. This application also claims priority under 35 U.S.C. § 119 of DE 102 59 302.7, filed Dec. 17, 2002, which is incorporated herein by reference in its entirety.

### BACKGROUND OF THE INVENTION

The present invention concerns a method for the quantitative and/or qualitative determination of germs. The present invention furthermore concerns an apparatus for the quantitative and/or qualitative determination of germs, in particular for carrying out the aforesaid method, and the use of that apparatus, in particular for preferably automated production and/or quality control.

Microbiological safety must be guaranteed for a large number of substances, raw materials, and products from a variety of sectors: industry, trades, household, health, food, etc.

It is important in this context that the nature and number of the various germs, for example bacteria and fungi, must be monitored within narrow limits.

Quantitative microbiological analysis techniques have already been used for quality assurance for some time. The basis of these methods is to propagate individual germs that occur in the material to be investigated, so that they are visible to the naked eye as streaks or colonies. It is standard practice to use for this purpose cultivation methods that propagate these germs either on solid nutrient substrates or in liquid nutrient solutions or media. Depending on the method and the type of germ, the execution of conventional cultivation methods for the detection of bacteria and fungi takes as long as several days.

According to the existing art, the procedure in the incubation methods is, in general, to inoculate nutrient media (typically culture dishes with agar-agar-based nutrient media) with the sample and cultivate them at (generally elevated) temperatures adapted to the respective germs for up to a week (e.g. in an incubator cabinet). Based on the growth and form of the resulting cultures, one skilled in the art can then deduce the nature and extent of the bacterial presence in the sample.

This technology has the critical disadvantage that only an undetermined fraction of the germs contained in the sample can be cultivated, and that the information is not available until a week later.

In order to solve the problems described above, a series of methods for accelerating microbial detection and enhancing sensitivity has already been developed in the past. These include microscopic methods that nonselectively or selectively stain the germs and correspondingly detect them, but also methods based on immunoassays as well as direct molecular-biology methods that amplify the genetic substance of the germs and then detect it by gel electrophoresis.

Attempts have been made for some time to reduce the detection time from several days to a few hours, or even less, using new so-called “fast detection methods.” “Fast detection methods” are in part already in use (impedance, bioluminescence, etc.), but a demand exists for faster and more direct methods, since even the “fast detection methods” already established are based on a time-dependent enrichment of biological material, so that 24 to 48 hours are still necessary for an analysis.

Optical fluorescence methods have, in the recent past, been increasingly replacing the conventional “fast detection methods” and cultivation processes. The direct epifluorescence filter technique (DEFT), for example, makes available for the first time a direct method that allows even a quantitative “living/dead” germ detection in less than an hour. This nonspecific optical fluorescence method has been known in academic basic research as a qualitative method for more than 25 years (see, for example, Pettipher et al., Appl. Environ. Microbiol. 44(4): 809-13, 1982), and since the early 1990s has increasingly become established in industrial applications (e.g. breweries, dairies, the food industry, etc.) as a quantitative investigation method (Hermida et al., J. AOAC Int. 83(6): 1345-1348, 2000 and Nitzsche et al., Brauwelt, No. 5, 177-178, 2000). A European specification also exists for the investigation of irradiated foods using a DEFT screening method (EN 13783: 2001 “Detection of irradiation of foods using epifluorescence filter technology/aerobic mesophilic germ count (DEFT/APC) screening method”).

Alternative selectively acting fluorescent dyes are offered by various manufacturers as laboratory kits for in vivo detection (e.g. by Molecular Probes and EasyProof Laborbedarf GmbH). Some suppliers (e.g. the Chemunex company) sell equipment systems; the systems offered by Chemunex, for example, are based either on the flow cytometry principle (L. Philippe, SÖFW-Journal 126, 28-31, 2000) that requires a 24-hour enrichment phase for the investigation of low germ levels, or on a microscopic filtration method that, however, does not permit “living/dead” differentiation (Wallner et al., PDA J. Pharm. Sci. Technol. 53(2): 70-74, 1999).

The so-called membrane filter microcolony fluorescence (MMCF) method (see e.g. J. Baumgart, Microbiological investigation of foods, Behr's . . . Verlag 1993, 3rd edition, pp. 98 ff.) provides for preparation of the sample, or of the germs present in the sample, on a membrane filter. A disadvantage here is that a time-consuming pre-enrichment of the germs must first occur, the membrane filter must be pretreated (moistened with special media, dimensioned, and dried) for subsequent epifluorescence microscopy, and the germ count must be made by counting the fluorescent-labeled colonies under an epifluorescence microscope or a UV lamp.

Although some of the technologies described above yield a result within a few hours, they are in general very complex in terms of the detection equipment required and the user knowledge that is necessary. For that reason, these methods have not hitherto become established to a sufficient extent for routine use. In addition, the individual methods have limitations in terms of specificity and sensitivity. Some of the above-described methods moreover possess too high a germ detection limit, so that prior cultivation often cannot be dispensed with.

In addition, the operating principles of conventional cultivation methods and “fast detection methods” with corresponding enrichment steps mean that some fundamental disadvantages exist:

Selection of the nutrient media plays a decisive role in terms of which microorganisms can be propagated. Selective nutrient media offer advantages here. Even these, however, can only propagate those microorganisms that have that physiological capability. According to the latest findings, however, only 5% of microorganisms can be cultivated. False-negative results therefore often occur with the conventional methods, even though the sample in fact contains germs.

In addition, the ability of analytical methods to provide information is limited by the time available. After completion of the enrichment time, all the germs must have propagated to the extent that they have become visible. Delayed growth due to unfavorable sampling or breeding conditions can thus result in erroneously negative results. Several days are therefore often needed to perform the conventional cultivation methods for the detection of bacteria and fungi, so that the microbiological results then often come too late to allow any regulating intervention in the production process.

With detection methods based on cultivation of the germs, only living germs capable of propagation can be detected. Often, however, the product preservation that exists has killed contaminants. The “dead” germs present in the product cannot be detected in this fashion, however, so that possible hygiene problems in production or packaging cannot be noted, or are detected only in the event of breakdowns, i.e. after a preservative failure.

The object underlying the present invention is therefore to make available a method of the kind cited initially that is suitable for the quantitative and qualitative determination of germs and, in particular, at least partly eliminates the disadvantages alluded to above; and a corresponding apparatus for carrying out such a method.

### BRIEF SUMMARY OF THE INVENTION

One aspect of the present invention is a method for quantitative and/or qualitative determination of germs in a liquid sample comprising the steps of: (1) passing the sample through a filter thereby depositing the germs on a major portion of the filter so that a minor portion of the filter is free of germ deposits; (2) applying a fluorescent label to at least a portion of the deposited germs; (3) determining the presence and/or the amount of labeled germs by fluorescent reflection photometry.

Another aspect of the present invention is an apparatus for quantitative and/or qualitative determination of germs in a liquid sample comprising: (1) a hollow cylindrical sample receptacle container having a first inlet opening at the top for receiving the sample, a second inlet opening at the top for receiving a fluorescent dye, a third inlet opening at the top for receiving a rinsing solution, a fourth inlet opening at the top for receiving compressed air; and a filter forming the bottom of the container wherein the filter is permeable to all substances except to germs which collect as a solid having an applied fluorescent label on the surface of the filter and wherein the outer rim of the filter is covered by the walls of the container so that the outer rim is free of deposited germs; (2) means for irradiating the solid germs with a light having a wavelength sufficient to cause the flourescent label to emit fluorescent light; (3) means for detecting the emitted fluorescent light; (4) means for measuring the intensity of the emitted fluorescent light; (5) determining the difference between the fluorescence intensity of the membrane region having labeled germs and the intensity of the rim region and calculating the amount of fluorescent labelled germs by comparing the intensity difference to a calibration curve.

### BRIEF DESCRIPTION OF THE SEVERAL VIEWS OF THE DRAWINGS

FIG. 1 is a schematic depiction an apparatus according to the invention for carrying out a method for quantitative and/or qualitative determination of germs in a sample.

FIG. 2 is a block diagram of the basic structure of the fluorescence reflection photometry detection system according to the present invention.

### DETAILED DESCRIPTION OF THE INVENTION

An essential concept of the present invention may therefore be seen in the fact that the germs present in the sample are first fluorescent-labeled, and the subsequent quantitative and/or qualitative evaluation or detection is accomplished by fluorescence reflection photometry. As will be explained in further detail herein, the use of fluorescence reflection photometry offers the great advantage of relatively simple detection or evaluation with little complexity, since the fluorescent-labeled germs require substantially no further preparation for that purpose. In particular, the time-consuming and labor-intensive method step of (pre-)enriching germs is omitted, i.e. the detection or evaluation according to the present invention by fluorescence reflection photometry directly supplies the “authentic” germ count, or the count present in the sample.

One special aspect of the method according to the present invention is therefore the combination of fluorescent labeling of germs, in particular microorganisms, using suitable fluorescent labels, and subsequent detection of the fluorescent-labeled germs using a simplified detection or evaluation method, i.e. by fluorescence reflection photometry, and a corresponding apparatus. Since the sample is only briefly exposed to irradiation in the fluorescence reflection photometry method, bleaching of the fluorescent labels, and therefore distortion of the measurement result, are efficiently prevented.

Detection or evaluation by fluorescence reflection spectrometry or fluorescence reflection photometry senses the radiation, or its intensity, emitted by reflection by the fluorescent-labeled germs upon irradiation at a corresponding wavelength, that radiation being correlated with the germ count present in the sample. (For further details about reflection photometry or reflection photometry methods, the reader may be referred, for example, to the relevant discussion in Römpp, Chemical dictionary, Thieme Verlag, 10th edition, Vol. 5, pp. 3756-3757, under “Reflection” and “Reflection spectroscopy,” and Vol. 4, pp. 3312 to 3314, under “Photometry,” including in each case the literature referenced therein, the entire contents of which are incorporated herein by reference.). The relatively laborious counting of colonies, and the germ enrichment preceding the counting, which are still necessary in the case of epifluorescence light microscopy as utilized, for example, in the context of the membrane filter microcolony fluorescence (MMCF) method, is thus avoided.

Fluorescence reflection photometers or spectrometers usable in the context of the method according to the present invention may readily be designed by one skilled in the art, proceeding from commercially available components commonly available for this purpose.

The phrase “labeling of at least a portion of the germs present in the sample” means that either a portion of a specific type of germ is labeled with a germ-specific fluorescent label or a portion of all of the germs is labeled with a germ-specific fluorescent label.

It is possible in accordance with the method according to the present invention, based on the measured value ascertained by fluorescence reflection photometry, to determine by suitable calibration the number of germs present in the sample (in the case of fluorescent labeling of all the germs present in the sample, all the germs present in the sample; and in the case of fluorescent labeling of only specific germs, the total number thereof). As will be further explained herein, the use of germ-specific fluorescent labels moreover makes possible a qualitative statement as to the presence of specific germs in the sample.

In general, in the context of the sample preparation carried out in method step (a), the fluorescent-labeled germs are applied onto the major portion of a membrane filter (e.g. a polycarbonate membrane filter) that preferably is porous. The membrane filter should in general be embodied in such a way that it retains the germs and/or is impermeable with respect to the germs. For that purpose, the size of the pores of the membrane filter should be selected so that the pore size is smaller than the germs present in the sample. As will be further explained herein, a minor portion or region of the membrane filter, generally the rim, should not be provided with or populated by germs, since this ensures a reference and internal calibration or standardization for each sample. Membrane filter materials in addition to polycarbonate that are also suitable according to the present invention are polytetrafluoroethylene (PTFE), polyester, and cellulose and cellulose derivatives, such as cellulose acetate, regenerated cellulose, nitrocellulose, or cellulose mixed esters. Membrane filters that are suitable according to the present invention are marketed, for example, by the Macherey-Nagel company (e.g. the “PORAFIL®” series).

The use of a membrane filter offers the great advantage that detection or evaluation by fluorescence reflection photometry can be accomplished directly on the membrane filter, in particular without further sample handling, preparation, transfer, or the like (i.e. in particular without pre-enrichment).

According to a particularly preferred embodiment of the method according to the present invention, the fluorescent-labeled germs are applied, in the context of the sample preparation performed in method step (a), onto a silicon microsieve. This is particularly advantageous because silicon microsieves have a particularly smooth and even surface, and germs present thereon can therefore be more easily detected. Such sieves are moreover relatively easy to clean, and they can be used repeatedly. Silicon microsieves furthermore possess good biocompatibility and good reflectivity. A further advantage of the microsieves may be seen in their rigid structure, which offers considerable advantages in terms of handling. The particularly homogeneous pore size is a further advantage that further increases the accuracy of selective filtration operations. The pore sizes of the microsieves to be used for the method according to the present invention should advantageously be between approximately 0.1 and 2 μm. Microsieves having pore sizes from 0.45 μm to 1.2 μm are particularly preferred. Sieves having pore sizes differing therefrom can, of course, also be used depending on the size of the germs to be determined, i.e. including those having pores smaller than 0.1 μm or larger than 2 μm.

Within the scope of the present invention, it is possible also to use silicon microsieves wherever membrane filters are used. Advantageous embodiments of the method or the apparatus according to the present invention having a membrane filter can thus also be implemented in each case using a silicon microsieve.

Advantageously, the fluorescent label used in the method according to the present invention is selected in such a way that it is membrane-transmissible with respect to the membrane filter used in the sample preparation performed in method step (a). This has the advantage that no background noise and no interference signals caused by excess fluorescent label occur in the context of detection or evaluation by fluorescence reflection photometry, and consequently a favorable signal-to-background ratio or signal-to-noise ratio is achieved.

Fluorescent labeling of the germs present in the sample in method step (a) of the method according to the present invention is performed in a manner well known in the art. This is common knowledge to one skilled in the art. For this purpose, for example, the germs to be labeled can be brought into contact with a solution or dispersion of the fluorescent label, present in an excess with respect to the germs that are present; the contact time must be sufficient to ensure complete fluorescent labeling of all the germs that are to be labeled in this method step (depending on the selection of the fluorescent label, for example, all the germs present in the sample or all the germs of only one or more germ types). After the actual fluorescent labeling, the excess fluorescent label can then be removed or separated from the fluorescent-labeled germs. This can be done, for example, in the case in which a porous membrane filter is used that is membrane-transmissible with respect to the fluorescent labels but impermeable with respect to the germs, by removing (e.g. by the application of overpressure or vacuum) the solution or dispersion of the excess fluorescent label through the porous membrane filter, and optionally then rinsing everything with water, buffer solutions, or other fluids, so that ultimately what remains on the membrane filter are only the fluorescent-labeled germs (together, if applicable, with the germs that have deliberately been left unlabeled). This then easily makes possible a subsequent detection or evaluation by fluorescence reflection photometry in method step (b) of the method according to the present invention.

If applicable, the sample preparation method step (a) can also encompass an inactivation and/or removal of germ-inhibiting and/or germ-killing substances or constituents (e.g. preservatives, surfactants, etc.) that may be present in the sample. This prevents the subsequent measurement results from being distorted by the fact that some of the germs are killed or inactivated by the germ-inhibiting or germ-killing substances or constituents during sample preparation or measurement, so that the germ count that is incorrectly too low is determined. This makes possible a determination of the germ count even in samples having germ-inhibiting and/or germ-killing substances or constituents (e.g. preservatives, surfactants, etc.), so that the method according to the present invention can also be used, for example, in surfactant and dispersion products.

The method step of inactivating or removing germ-inhibiting or germ-killing substances or constituents that may be present in the sample, performed only as applicable depending on the type of sample, is advantageously performed prior to fluorescent labeling, preferably directly after sampling or at the very beginning of the sample preparation performed in method step (a) of the method according to the present invention; this ensures that the germ-inhibiting or germ-killing substances, constituents, ingredients, and the like will have been able to effect substantially no change in the germ count present in the original sample. It is equally possible, although less preferred, to perform the inactivation or removal of germ-inhibiting or germ-killed substances or constituents that may be present in the sample after fluorescent labeling. It is likewise possible to perform fluorescent labeling and the inactivation or removal of the germ-inhibiting or germ-killing substances or constituents simultaneously.

The method step of inactivating or removing any germ-inhibiting or germ-killing substances or constituents that may be present in the sample, performed only as applicable depending on the type of sample, is accomplished in a manner well known in the art such as, for example, as set forth by Stumpe et al., “Chemoluminescence-based direct detection of microorganisms: a report on experience in the food and cosmetics industry,” on pages 317 to 323 of the conference proceedings “HY-PRO 2001, Hygienische Produktionstechnologie/Hygienic Production Technology,” 2nd International Conference and Exposition, Wiesbaden May 15-17, 2001, and the literature indicated in this contribution; the entire contents of this contribution, including the contents of the literature cited therein, is hereby incorporated by reference. This can generally be accomplished by bringing the sample that is to be analyzed into contact with a suitable inactivation and/or conditioning solution. Such inactivation or conditioning solutions are well known to those skilled in the art (e.g. aqueous TLH [Tween/lecithin/histidine] conditioning solution). Aqueous inactivation or conditioning solutions that are suitable according to the present invention can also contain, for example, in addition to TLH (e.g. polysorbate 80=Tween 80, soy lecithin, and L-histidine), buffer substances (e.g. phosphate buffers such as hydrogenphosphate and/or dihydrogenphosphate), further salts (e.g. sodium chloride and/or sodium thiosulfate), and tryptone (peptone from casein).

An inactivation or conditioning solution that is particularly suitable according to the present invention has the following composition:

| | | |
|---|---|---|
| Tryptone | 1.0 | g |
| Sodium chloride | 8.5 | g |
| Sodium thiosulfate pentahydrate | 5.0 | g |
| 0.05 M phosphate buffer solution | 10 | ml |

| | |
|---|---|
| TLH in water | to make 1000 ml |

The “TLH in water” usable according to the present invention has, in particular, the following composition:

| | |
|---|---|
| Polysorbate 80 (Tween 80) | 30.0 g |
| Soy lecithin | 3.0 g |
| L-histidine | 1.0 g |
| Deionized water | to make 1000 ml |

The phosphate buffer solution usable according to the present invention has, in particular, the following composition:

| | |
|---|---|
| Potassium dihydrogenphosphate | 6.8045 g |
| Dipotassium hydrogenphosphate | 8.709 g |
| Deionized water | to make 1000 ml. |

The selection of fluorescent label(s) is not critical. Fluorescent labels well known from the existing art can be used here depending on the application and the type of germs, provided they are suitable for use within the scope of the method according to the present invention.

The term “fluorescent label” is to be understood very broadly within the scope of the present invention, and means in particular any fluorescent label which is embodied in such a way that it enters into an interaction with the germs, for example binds to the germs, in particular to their cell wall (envelope) and/or nucleic acid, and/or is taken up by the germs, in particular is metabolized and/or enzymatically converted.

The fluorescent label used can be, for example, a non-germ-specific fluorescent label or a mixture of non-germ-specific fluorescent labels. This makes possible relatively economical fluorescent labeling of all the germs present in the sample, and thus a relatively rapid determination of the total germ count in the sample.

In particular in the case in which only specific germs are to be qualitatively and quantitatively sensed in selective fashion, a germ-specific fluorescent label or a mixture of different germ-specific fluorescent labels can be used as the fluorescent label.

Similarly, a mixture of non-germ-specific and germ-specific fluorescent labels can be used as the fluorescent label.

For example, a fluorescent label entering into interaction with living germs can be used as the fluorescent label. Similarly, a fluorescent label entering into interaction with “dead” germs can also be used as the fluorescent label.

It is likewise possible to use as the fluorescent label a mixture of fluorescent labels entering into interaction with living germs, and fluorescent labels entering into interaction with “dead” germs. A living/dead differentiation of the germs present in the sample can thereby be achieved. Such mixtures are well known in the art (see e.g. Stumpe et al., loc. cit., and the system cited therein of EasyProof Laborbedarf GmbH, Voerde).

The aforementioned label system of EasyProof Laborbedarf GmbH was originally introduced for the brewing industry (Eggers et al., Brauindustrie 6, 34-35, 2001). Here a non-fluorescing preliminary stage (i.e. a precursor) of a fluorescent label is taken up into an intact microbial cell and is converted by enzymatic activity (esterase) within the cell (cytoplasm) into a fluorescing compound (green color=detection as living); to allow this procedure to occur, an intact cell membrane having a membrane potential must be present. Detection of “dead” cells is accomplished by the introduction of a specific fluorescent dye into the cell's DNA. This incorporation can in turn occur only in cells that have a defective cell membrane (red color=detection as dead). Because the two reactions are based on different principles, the results are independent of one another. This labeling technique does not damage the cells, so that the current microbiological status of the sample can be determined in the context of the evaluation.

The fluorescent labels usually used for germ labeling in epifluorescence microscopy or in the direct epifluorescence filter technique (DEFT) or in the membrane filter microcolony fluorescence (MMCF) method can also, for example, be used as fluorescent labels in the method according to the present invention.

For example, a fluorescent dye or a precursor of such a fluorescent dye from which the fluorescent dye is generated by interaction with the germs, in particular by metabolization and/or enzymatic conversion, can be used as the fluorescent label.

Examples of such precursors of fluorescent dyes are described, for example, in U.S Pat. No. 5,089,395 and in EP 0 443 700 A2, the entire respective disclosure contents of each of which is hereby incorporated by reference).

Examples of fluorescent dyes usable according to the present invention as fluorescent labels are, without limitation, for example 3,6-bis[dimethylamino]acridine (acridine orange), 4′,6-diamido-2-phenylindole (DAPI), 3,8-diamino-5-ethyl-6-phenylphenanthridinium bromide (ethidium bromide), 3,8-diamino-5-[3-(diethylmethyammonio)propyl]-6-phenylphenanthridinium diiodide (propidium iodide), rhodamines such as rhodamine B and sulforhodamine B, and fluorescein isothiocyanate. For further examples the reader may also refer to EP 0 940 472 A1 or to Molecular Probes' Handbook of Fluorescent Probes and Research Chemicals, 5th edition, Molecular Probes Inc., Eugene, Oreg. (P. R. Haugland, editor, 1992), the entire respective disclosure contents of which are herewith incorporated by reference. Reference may also be made to the relevant chemical catalogs (e.g. “Fluorescent Labeling Reagents” catalog of biochemicals and reagents for life science research, of the Sigma-Aldrich company, 2002/2003 edition).

It is also possible to use as fluorescent labels in the method according to the present invention, for example, nucleic acid probes (e.g. germ-specific nucleic acid probes) that in turn are fluorescent-labeled, in particular with a fluorescing group or a fluorescing molecule. The fluorescing group or fluorescing molecule can be bound, for example, covalently or otherwise to the nucleic acid probe. The nucleic acid probe used according to the present invention as a fluorescent label can be, for example, a fluorescent-labeled nucleic acid oligonucleotide or polynucleotide or a fluorescent-labeled DNA probe or RNA probe. For stability reasons, DNA probes are generally preferred according to the present invention.

Examples of nucleic acid probes usable according to the present invention as fluorescent labels are, for example, the probes recited in WO 01/85340 A2, WO 01/07649 A2, and WO 97/14816 A1, the entire respective disclosure contents of which are incorporated herein by reference.

For example, the nucleic acid probes usually utilized in fluorescence in situ hybridization (FISH) for labeling (DNA or RNA labeling) can be used as nucleic acid probes. For further details relevant thereto, the reader may refer to the Römpp Dictionary of biotechnology and genetic engineering, 2nd edition, Georg Thieme Verlag Stuttgart, pp. 285-286, under “FISH,” and to the literature cited therein, and to WO 01/07649 A2, the entire respective disclosure contents of which are herewith incorporated by reference.

It is likewise possible to use as the fluorescent label an, in particular, germ-specific antibody that in turn is fluorescent-labeled, in particular with a fluorescing group or a fluorescing molecule, in particular such that the fluorescing group or fluorescing molecule can be bound covalently or otherwise to the antibody.

The quantity or concentration of fluorescent labels that are used will be adapted by the person skilled in the art to the particular circumstances of the individual case. This is entirely common practice for such a person. In the context of a living/dead differentiation of the germs present in the sample, for example, a suitable mixing ratio of “living dye” to “dead dye” should be selected for good germ staining simultaneously with weak “background staining”; the selection in the individual case is within the specialized ability of one skilled in the art.

With the method according to the present invention, the detection limit with respect to the germs to be determined is generally≦100 colony-forming units (CFUs) per milliliter of sample volume, preferably≦10 colony-forming units (CFUs) per milliliter of sample volume. The method according to the present invention therefore dispenses with any pre-enrichment. The low detection limit is critically important, for example, for compliance with certain guidelines or specifications. According to CTFA guidelines for cosmetic raw materials, for example, with a much higher germ count limit (e.g. 10² to 10³ CFUs/ml), a time-consuming and cost-intensive test for the present of certain problem germs, i.e. pathogenic germs, must be performed.

With the method according to the present invention, it is possible in general to determine germ counts in the range from approximately 10 CFUs per milliliter of sample volume, or even less, to approximately 10⁸ CFUs per milliliter of sample volume. For quantitative evaluation purposes, above a certain germ count (generally above approx. 10² CFUs per milliliter of sample volume), the sample should first be correspondingly (i.e. suitably) diluted.

The method according to the present invention is suitable in principle for the determination of any germs, in particular pathogenic germs of all types (e.g. microorganisms of all types, in particular unicellular microorganisms such as bacteria and fungi, e.g. yeasts or molds).

The method according to the present invention is suitable in principle for quantitative and/or qualitative determination of germs in any products (e.g. media, matrices, solutions, etc.), preferably filterable, in particular liquid and/or pourable products. In the case of solid products or those not filterable as such, they must be converted during sample preparation into a form accessible to the method according to the present invention; this is done using methods well known in the art, for example by conversion into a solution or dispersion, by comminution, extraction, etc.

The method according to the present invention is suitable, for example, for quantitative and/or qualitative determination of germs in foodstuffs, surfactant-containing products such as washing and cleaning agents, surface treatment agents, dispersion products, cosmetics, hygiene products and personal care products, pharmaceuticals, adhesives, coolant lubricants, coatings and (coating) coagulations, as well as raw materials and starting materials for the aforesaid products.

The method according to the present invention is therefore suitable for all types of possible raw materials, intermediaries, and end products from the various sectors, for example foods, branded goods, cosmetics, adhesives, coolant lubricants (e.g. oily coolant lubricant emulsions); process fluids of industrial systems, etc., with the limitation that it should be possible to separate the germs to be detected using a separation method, such as filtration or sedimentation. It is also immaterial in this context whether the products exist in solid or liquid form.

Because of its relatively simple execution and the particular combination of method steps, the method according to the present invention is particularly suitable for automated execution (e.g. in the context of production and/or quality control). In addition to production and/or quality control (e.g. during the packaging of liquid surfactant products, dispersions, preserved products, etc.), the method according to the present invention is also suitable, for example, for the investigation of malfunctions or contamination instances to determine germ status, or also for the evaluation of product sanitizing measures, or also for the optimization or checking of facility cleaning actions (e.g. in facilities for producing preserved products), for example in the context of cleaning in place (CIP) and sterilization in place (SIP) processes.

The method according to the present invention is usually carried out as follows: The sample having the germs to be quantitatively and/or qualitatively determined is introduced into a suitable sample vessel, which should be sealable in germ-free fashion and the bottom of which is equipped with a generally round membrane. The membrane filter rests with its outer rim on the sample vessel, so that the outer, concentric rim is not populated by germs. If a sample comprising germ-inhibiting or germ-killing substances or constituents (e.g. preservatives or surfactants) is to be investigated, firstly an inactivation and/or removal of those substances or constituents is performed, by bringing the sample into contact with a suitable inactivation and/or conditioning solution, specifically for a period of time sufficient to allow inactivation and/or removal of those substances or constituents. The inactivation and/or conditioning solution is removed through the membrane filter using overpressure or vacuum. Excess or remaining inactivation and/or conditioning solution is then removed as necessary through the membrane filter by washing once or several times with water, usually by application of an overpressure or vacuum, so that the wash water is also removed in simple fashion. This is followed by labeling of at least some of the germs present in the sample by means of at least one fluorescent label. For that purpose the germs can be brought into contact with, for example, a solution or dispersion of the fluorescent label, for a time sufficient to label the germs. The excess solution or dispersion of the fluorescent label is then removed through the membrane filter by once again applying an overpressure or vacuum. Lastly, if applicable, the sample can be subjected to a single or multiple rinse with water, buffer solutions, or other liquids in order to remove excess fluorescent label. After withdrawal of the water through the membrane filter by the application of overpressure or vacuum, the membrane filter can then finally be detached from the sample vessel, yielding a membrane filter populated with fluorescent-labeled germs, the outer rim of which is free of germs. This can be sent on immediately, i.e. generally without further preparation or treatment of the sample or the filter, for fluorescence reflection photometry. In the context of the measurement by fluorescence reflection photometry, the membrane filter populated with fluorescent-labeled germs is then irradiated with light of a suitable wavelength, being, so to speak, scanned in the process. The measured value that is ascertained is correlated with the germ count on the membrane filter or in the sample. The following washing agent products, of various viscosities, were investigated using the apparatus according to the present invention:

| | | Viscos- | Temper- | Rotation | |
|---|---|---|---|---|---|
| Product | | ity | ature | rate | Spindle |
| category | Viscosimeter | (mPa) | (° C.) | (min⁻¹) | no. |
| Delicate fabric | Brookfield | 200 | 20 | 30 | 31 |
| washing agent | LV | | | | |
| Conditioner | Brookfield | 175 | 20 | 20 | 31 |
| | LVDV II+ | | | | |
| Conditioner | Brookfield | 150 | 20 | 20 | 31 |
| | LVDV II+ | | | | |
| Hand | Brookfield | 400 | 20 | 30 | 31 |
| dishwashing | LVDV II+ | | | | |
| liquid | | | | | |
| Hand | Brookfield | 1500 | 20 | 30 | 31 |
| dishwashing | LVDV II+ | | | | |
| liquid | | | | | |
| Liquid heavy- | Brookfield | 1800 | 20 | 20 | 3 |
| duty cleaning | RV | | | | |
| agent | | | | | |
| Liquid heavy- | Brookfield | 1500 | 20 | 20 | 2 |
| duty cleaning | RV | | | | |
| agent | | | | | |
| Liquid heavy- | Brookfield | 2750 | 20 | 20 | 3 |
| duty cleaning | RVDV II+ | | | | |
| agent | | | | | |
| Liquid heavy- | Brookfield | 2750 | 20 | 20 | 3 |
| duty cleaning | RVDV II+ | | | | |
| agent | | | | | |

A typical process sequence for the method according to the present invention in the context of automatic execution contains, for example, the fact that the user places a defined quantity (e.g. 1 ml) of a sample into a predefined sample vessel that is sealed in germ-free fashion and contains a condition or inactivation medium and a membrane filter at the outlet. Starting the measurement program causes the sample to be shaken and thermostatically controlled to a suitable temperature (e.g. 37° C.) that depends on the type of germs. After a defined time, the medium is filtered off through the membrane filter. One or more washing steps with germ-free water, buffer solutions, or other liquids then automatically follow in order to wash out substances present in the sample, followed by labeling with a fluorescent label. Depending on requirements, labeling can be accomplished with a non-specific fluorescent label that attaches to all the germs present in the sample (e.g. to nucleotide fragments), or with a label that uses the DEFT method to allow a differentiation of all living and “dead” microorganisms, or in the third case with a fluorescent label based on FISH technology, which makes possible selective staining of individual microorganisms using gene-labeled fluorescent probes. Subsequent to the labeling step, excess labeling solution is once again automatically washed out with water, so that after completion of the automatic protocol, fluorescent-labeled microorganisms are present on the membrane filter. The intensity of the respective fluorescence, and therefore the number of microorganisms, is then automatically determined using a fluorescence reflection photometer or spectrometer. In this, the filter membrane is irradiated with light of a suitable wavelength, and the fluorescent light emitted by the labeled microorganisms is detected in correspondingly wavelength-resolved fashion. Subsequent evaluation compares the intensity in the rim region of the membrane, which should not be populated with microorganisms, with the intensity in the region having labeled microorganisms, and calculates the number of germs present in the sample on the basis of a stored calibration.

A number of advantages are associated with the method according to the present invention; the essential ones are discussed herein, albeit not in limiting fashion.

One advantage of the method according to the present invention may be seen in the fact that it can be performed in automated fashion. Complete automation of the entire procedure means that the method can be carried out more easily, more quickly, and more reproducibly. This yields advantages in terms of cost, personnel requirements, and sensitivity. Good reproducibility when carrying out the investigations is likewise very advantageous.

A further advantage of the method according to the present invention is that it does not require an epifluorescence microscope. Replacement of the epifluorescence microscope used according to the existing art with the simplified evaluation and detection method and system (i.e. using fluorescence reflection photometry) reduces the outlay required of the user in terms of work and capital equipment, and evaluation of the samples can be performed entirely automatically (e.g. using a corresponding algorithm). Radiation impact is also decreased.

Yet another advantage of the method according to the present invention is the use of standardized or conventional components: the entire system required for carrying out the method according to the present invention is integrated in such a way that numerous standardized or conventional components (vessels, media, filters, etc.) can be used, thus reducing operator effort and enhancing the reliability of the method.

Another advantage of the method according to the present invention is its simple detection and evaluation: the method according to the present invention can be carried out, for example, in a suitable sample vessel, so that the labeled germs are prepared on a suitable filter membrane. Selection of an appropriate membrane size (e.g. 8 mm diameter) and a concentric rim not populated with germs allows referencing and internal standardization for each sample.

Yet another advantage of the method according to the present invention is the speed with which the method according to the present invention is carried out: the method according to the present invention permits a determination of germ count after a period of only a few (approx. 3 to 5) minutes, depending on the type of germs and their quantity. Conventional culture methods, on the other hand, require up to several days.

The high sensitivity of the method according to the present invention is also particularly advantageous: the method according to the present invention allows the determination of germs even at high dilutions. Accelerated culture methods are not sufficiently sensitive for a detection limit of 10 CFUs per milliliter of sample volume.

A further advantage of the method according to the present invention is the fact that the fluorescent-labeled germs are exposed to irradiation for only a relative short time, since the measured values can be acquired relatively quickly by fluorescence reflection photometry. As a result, any “bleaching” of the fluorescent label, and therefore distortion of the measurement result, is almost completely avoided. This also prevents the killing of living germs, which is of critical importance especially in the context of living/dead differentiation.

“Scanning” of the sample (or more precisely of the membrane filter populated with fluorescent-labeled germs) in the context of the method according to the present invention or in the context of evaluation by fluorescence reflection photometry yields further advantages: On the one hand, scanning allows large areas to be excited. On the other hand, especially as compared with a conventional fluorescence microscope having a limited sensed area, a high single-point excitation intensity is achieved along with a short comparison. Homogeneous illumination of the samples as a result of scanning is critically important especially in the context of intensity measurements.

Lastly, the user-friendliness of the method according to the present invention must be seen as a further advantage. The entire process of determining the germ load in a sample involves, in a context of automatic execution, simply adding the sample and starting the sequence. The user then receives a numerical value for the germ load. The effort involved in sample preparation and measurement is minimal. The system can thus also, ideally, be incorporated into process systems for monitoring, assuring, and documenting quality.

According to a further, second aspect of the present application, the present invention also concerns an apparatus, as described in Claim 28, for quantitative and/or qualitative determination of germs in a sample in accordance with a method with sample preparation and subsequent detection and/or evaluation, a labeling of at least some of the germs present in the sample using at least one fluorescent label being performable by means of the apparatus in the course of sample preparation, and detection and/or evaluation being performable utilizing the fluorescent label, in particular for carrying out a method as defined in any of the preceding method claims.

Further advantageous embodiments of the apparatus according to the present invention are the subject matter of the dependent apparatus claims (Claims 29 to 36). The statements made with reference to the method according to the present invention apply correspondingly to the apparatus according to the present invention.

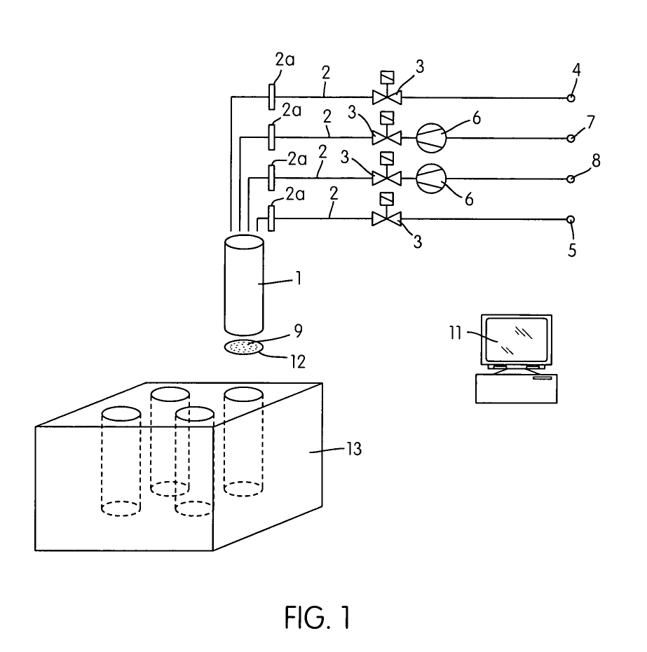

In the drawings, FIG. 1 schematically depicts an apparatus for carrying out a method for quantitative and/or qualitative determination of germs in a sample. The cornerstone of this apparatus is a sample receptacle container 1. This sample receptacle container 1, as schematically indicated, is connected in the apparatus via various lines 2 to control valves 3 for venting 4 and compressed air 5, and via pumps 6 to connectors for dye 7 (“fluorescent label”) and rinsing solution 8. Sterile filters 2a are integrated into each line 2.

FIG. 1 schematically depicts the relationships that have already been explained. The manner of operation of such an apparatus logically follows the procedure explained with reference to the method claims.

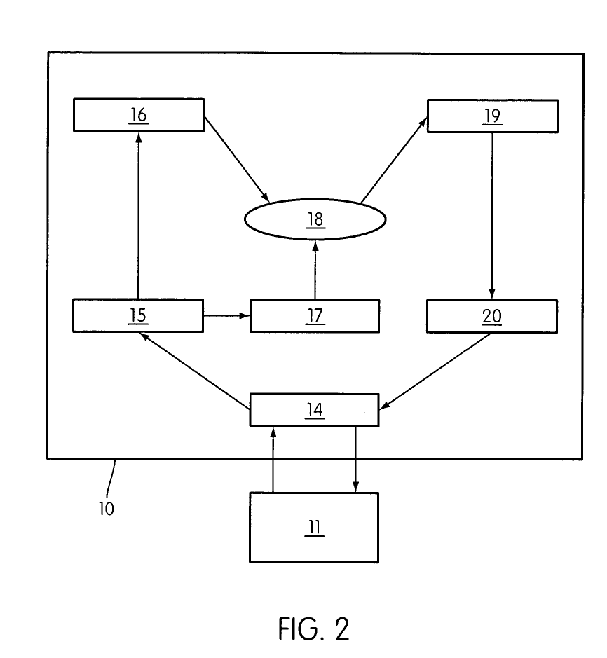

Arranged at an outlet of sample receptacle container 1—at the bottom of sample receptacle container 1, in the preferred exemplifying embodiment depicted—is a membrane filter 9 that is indicated in the exemplifying embodiment depicted as a simple circular disk. The latter is embodied so that it retains the germs that are to be detected and/or is impermeable with respect to the germs to be detected. Also provided is a detection system 10 that is configured to perform a measurement by fluorescence reflection photometry, and on which is positionable, for purposes of detection and/or evaluation, sample receptacle container 1 with membrane filter 9, or preferably membrane filter 9 removed from sample receptacle container 1. Analysis of the germ-populated membrane 9 in fluorescence reflection photometry detection system 10, the general construction of which is depicted in FIG. 2, is accomplished in the exemplifying embodiment depicted using a computer-controlled control and/or evaluation device 11 that has already been indicated in FIG. 1.

FIG. 1 also shows, in indicative fashion, membrane filter 9 on the bottom of sample receptacle container 1. This means that in the exemplifying embodiment depicted in FIG. 1, membrane filter 9 can be removed from herein from sample receptacle container 1 in order to be passed on to detection system 10. The specific appearance of the arrangement here is left to the design capabilities of one skilled in the art.

It is particularly advantageous if membrane filter 9 is a membrane filter having pores, in particular a polycarbonate membrane filter; and if, in particular, the size of the pores of membrane filter 9 is smaller than the size of the germs to be determined that are present, or expected to be present, in the sample.

It is very particularly preferred if a silicon microsieve is used instead of membrane filter 9. The advantages arising from the use of silicon microsieves have already been discussed in detail above. It will be mentioned at this juncture as well, however, that a silicon microsieve can be used in general instead of a membrane filter in the apparatus according to the present invention.

In terms of both the arrangement within sample receptacle container 1 and the separation of membrane filter 9 from sample receptacle container 1 for the purpose of detecting the labeled germs, it is advisable to work with a reference surface so that each sample can be standardized self-sufficiently. According to a preferred embodiment, provision can be made for that purpose for membrane filter 9 arranged in sample receptacle container 1 to have a region 12, in particular a rim, that cannot or cannot practically be populated with germs during sample preparation, and that serves as a reference surface when detection and/or evaluation is carried out. The region or rim 12 not populated with labels allows internal standardization of the sample itself on membrane filter 9.

With regard to the dimensioning of membrane filter 9 and of sample receptacle container 1, for the application indicated it is advisable that membrane filter 9 have a diameter effective for filtration of approximately 5 to approximately 25 mm, in particular approximately 6 mm to approximately 12 mm, preferably approximately 8 mm to approximately 10 mm.

It has already been alluded to above that membrane filter 9 retains the germs, i.e. that the germs labeled with the fluorescent label remain behind on the upper side of membrane filter 9. It is recommended for that purpose to select the fluorescent label in such a way that it is membrane-transmissible with respect to membrane filter 9, so that a favorable signal-to-noise ratio is achieved in detection system 10. For handling of the sample in sample receptacle container 1, isolation of the fluorescent-labeled (“marked”) germs on membrane filter 9 must be effected. In terms of apparatus, what is advisable for this purpose is the use of a drip-off device, a suction device (vacuum), and/or an expulsion device to drip off and collect a sample fluid and/or receiving fluid for the fluorescent label and/or rinsing fluid present above membrane filter 9. The exemplifying embodiment depicted in FIG. 1 shows, in this context, a variant in which expulsion is accomplished using compressed air 5.

Lastly with regard to apparatus, FIG. 1 also shows that the apparatus comprises a thermostat device 13 for thermostatic control of sample receptacle container 1. Visible here is thermostat device 13 having a total of four receptacle openings, so that a total of four sample receptacle containers 1 can be simultaneously thermostatically controlled to the normally desired temperature, which depends on the target germs (e.g. 37° C.).

FIG. 2 depicts the basic structure of the fluorescence reflection photometry detection system 10 that is used according to the present invention. Evaluation device 11 controls a central controller 14 that forwards the measurement parameters to an electronic control system 15 for excitation optical system 16 and a positioning stage 17. The measured specimen, i.e. in this case membrane filter 9 populated with labeled germs, is located on positioning stage 17. Excitation light emitted from excitation optical system 16 onto measured specimen 18 is reflected, as fluorescent light, to a detection optical system 19. The detected signal is conveyed to an electronic measurement system 20 that in turn feeds into controller 14. The use of a simplified fluorescence reflection photometry detection system 10 instead of a complex epifluorescence microscope reduces the user's capital expenditure.

According to a further, third aspect of the present Application, the present invention furthermore concerns the use according to the present invention of the apparatus according to the present invention as described above, constituting the subject matter of the use claims (Claims 37 to 39).

Further embodiments, modifications, variations, and advantages of the present invention will be readily apparent to and achievable by one skilled in the art from a reading of the description, without departing from the context of the present invention.

What is claimed is:

1. A method for quantitative and/or qualitative determination of germs in a liquid sample comprising the steps of: (1) passing the sample through a filter thereby depositing the germs on a major portion of the filter so that a minor portion of the filter is free of germ deposits; (2) applying a fluorescent label to at least a portion of the deposited germs; (3) determining the presence and/or the amount of labeled germs by fluorescent reflection photometry.

2. The method of claim 1 wherein the filter is a membrane filter selected from the group consisting of polycarbonate, PTFE, polyester, cellulose, a cellulose derivative, and cellulose mixed esters.

3. The method of claim 1 wherein the membrane filter is a polycarbonate membrane filter.

4. The method of claim 3 wherein the cellulose derivative is cellulose acetate, regenerated cellulose, or nitrocellulose.

5. The method of claim 1 wherein the pore size of the membrane filter is smaller than the deposited germs.

6. The method of claim 1 wherein the fluorescent label is selected in such a way that it is transmissible through the membrane filter.

7. The method of claim 1 wherein the fluorescent label is chosen so that it binds to the cell wall of the germ, a nucleic acid, or is metabolized, or enzymatically converted.

8. The method of claim 1 wherein the fluorescent label is a non-germ-specific fluorescent label or a mixture of non-germ-specific fluorescent labels.

9. The method of claim 1 wherein the fluorescent label is a mixture of non-germ-specific and germ-specific fluorescent labels.

10. The method of claim 1 wherein the fluorescent label is a mixture comprised of a fluorescent label that interacts with living germs and a fluorescent label that interacts with dead germs whereby a living/dead differentiation of the germs present in the sample is determined.

11. The method of claim 1 wherein the fluorescent label is a fluorescent dye or a precursor of a fluorescent dye.

12. The method of claim 11 wherein the fluorescent dye is generated by metabolization and/or enzymatic conversion of the fluorescent dye precursor by the germs.

13. The method of claim 11 wherein the fluorescent dye is selected from the group of: 3,6-bis[dimethylamino]acridine (acridine orange), 4′,6-diamido-2-phenylindole (DAPI), 3,8-diamino-5-ethyl-6-phenylphenanthridinium bromide (ethidium bromide), 3,8-diamino-5-[3-(diethylmethyammonio)propyl]-6-phenylphenanthridinium diiodide (propidium iodide), rhodamine B, sulforhodamine B, and fluorescein isothiocyanate.

14. The method of claim 1 wherein the fluorescent label is a fluorescent-labeled, germ-specific nucleic acid probe.

15. The method of claim 14 wherein the nucleic acid is oligonucleotide or polynucleotide.

16. The method of claim 15 wherein the nucleic acid probe is a fluorescent-labeled DNA or RNA probe.

17. The method of claim 1 wherein the fluorescent label is a fluorescent-labeled, germ-specific antibody.

18. The method of claim 1 wherein the detection limit of the germs is ≦100 colony-forming units (CFUs) per milliliter of sample volume.

19. The method of claim 18 wherein the detection limit is ≦10 colony-forming units (CFUs) per milliliter of sample volume.

20. The method of claim 1 wherein the germs are pathogenic germs.

21. The method of claim 20 wherein the pathogenic germs bacteria and fungi.

22. The method of claim 1 wherein the method is used for quantitative and/or qualitative determination of germs in foodstuffs and surfactant-containing products.

23. The method of claim 22 wherein the surfactant-containing products are washing and cleaning agents, surface treatment agents, dispersion products, cosmetics, hygiene products, personal care products, pharmaceuticals, adhesives, coolant lubricants, coatings and coating coagulations.

24. The method of claim 1 wherein the filter is a silicon microsieve.

25. A method for quantitative and/or qualitative determination of germs in a liquid sample comprising the steps of: (1) providing a liquid sample comprised of germs and germ-inhibiting and/or germ-killing substances; (2) removing the germ-inhibiting and/or germ-killing substances from the sample; (3) passing the sample from step (2) through a membrane filter or a silicon microsieve thereby depositing the germs on a major portion of the filter so that a minor portion of the filter is free of germ deposits; (4) applying a fluorescent label to at least a portion of the deposited germs; (5) determining the presence and/or the amount of labeled germs by fluorescent reflection photometry.

26. An apparatus for quantitative and/or qualitative determination of germs in a liquid sample comprising: (1) a hollow cylindrical sample receptacle container having a first inlet opening at the top for receiving the sample, a second inlet opening at the top for receiving a fluorescent dye, a third inlet opening at the top for receiving a rinsing solution, a fourth inlet opening at the top for receiving compressed air; and a filter forming the bottom of the container wherein the filter is permeable to all substances except to germs which collect as a solid having an applied fluorescent label on the surface of the filter and wherein the outer rim of the filter is covered by the walls of the container so that the outer rim is free of deposited germs; (2) means for irradiating the solid germs with a light having a wavelength sufficient to cause the flourescent label to emit fluorescent light; (3) means for detecting the emitted fluorescent light; (4) means for measuring the intensity of the emitted fluorescent light; (5) determining the difference between the fluorescence intensity of the membrane region having labeled germs and the intensity of the rim region and calculating the amount of fluorescent labelled germs by comparing the intensity difference to a calibration curve.

27. The apparatus of claim 26 wherein the filter is a membrane filter or a silicon microsieve.

28. The apparatus of claim 27 wherein the filter is a porous polycarbonate membrane filter.

29. The apparatus of claim 27 wherein the size of the pores of the membrane filter or silicon microsieve is smaller than the size of the deposited germs.

30. The apparatus of claim 26 wherein the filter has a diameter of from approximately 5 mm to approximately 25 mm.

31. The apparatus of claim 30 wherein the diameter is from approximately 6 mm to approximately 12 mm.

32. The apparatus of claim 30 wherein the diameter is from approximately 8 mm to approximately 10 mm.

33. The apparatus of claim 26 further comprising a thermostat for thermostatic control of the sample receptacle container.

34. The apparatus of claim 26 wherein the liquid sample is selected from the group consisting of foodstuffs, washing and cleaning agents, surface treatment agents, dispersion products, cosmetics, hygiene products and personal care products, pharmaceuticals, adhesives, coolant lubricants, coatings and coating coagulations.
